(19) United States
(12) Patent Application Publication
(10) Pub. No.: US 2006/0078948 A1
(43) Pub. Date: Apr. 13, 2006

(54) Analytical method, analytical instrument, microarray and immunoassay of biological samples

(76) Inventors: Takuya Matsui, Hitachinaka (JP); Katsuhiro Kanda, Hitachinaka (JP)

Correspondence Address: MCDERMOTT WILL & EMERY LLP, 600 13TH STREET, N.W., WASHINGTON, DC, 20005-3096, US

(21) Appl. No.: 11/239,467

(22) Filed: Sep. 30, 2005

(30) Foreign Application Priority Data
Oct. 6, 2004 (JP) 2004-293420

(51) Int. Cl. G01N 33/542; C12M 1/34

(52) U.S. Cl. 435/7.9; 435/287.2

(57) ABSTRACT

An object of the present invention is to provide an analytical method and an analytical instrument with high capability of quantitative determination and reproducibility which can be used for detecting the minor difference between expression levels and measuring the protein concentration, for example. An analytical method for a biological sample is disclosed, wherein two or more labels are present in the same area, and the signal intensity of one specific label is used to normalize the signal intensity of other labels. An analytical instrument, a microarray, and an immunoassay are also disclosed.

### BACKGROUND OF THE INVENTION

The present invention relates to a method for detecting genes using microarray and the like, and to an analytical method, an analytical instrument, a microarray and an immunoassay which can be used as the method for detecting interaction between proteins in biological samples.

### PRIOR ART

The microarray is a device in which from several hundreds to several ten thousand spots of DNAs or proteins are arranged and fixed onto a substrate such as a slide glass, silicone and the like, and this technology makes the work requiring from several hundreds to several ten thousand experiments by the conventional technology to be carried out simultaneously altogether. It becomes an important technology in the field of medical drug in life science where high throughput is demanded, such as searching for genes related to diseases in drug research and the like. Further, a microchip is the microarray in which channels and the like are formed.

The human genome analyses predict that about 30,000 genes exist in human and over 100,000 proteins are produced from these genes. A simple method for selecting a target protein from these 100,000 proteins, that is screening, is expected to bring in great development in the field of drug development. For example, a practical protein chip, in which a plurality of proteins are fixed onto a substrate, is very useful as a low cost and simple screening technology.

The microarray, although it can accommodate a large number of samples, has drawbacks such as poor capability of quantitative determination and reproducibility. The reasons for the drawbacks include cross hybridization due to coagulation of probes on the surface of the microarray, poor efficiency of labeling of samples, cross-contamination of samples, unstability of laser, warping of slide glass, unevenness of linker coating which is a fixation agent for DNA/protein, inaccuracy of spotting of an arrayer, difference of amount of spotting by the arrayer and the like. Thus, in the presence of these problems, the same operation results in poor capability of quantitative determination and reproducibility.

To solve these problems, the correction of the spot signal intensity may be possible by applying a plurality of positive control spots having a constant level of signal intensity around the spot needed to be calibrated assuming that these signal intensity is constant. However, the correction does not necessarily reflect the information of the position needed to be calibrated correctly, and if the spot needed to be calibrated has a specific defect, it may not be possible to calibrate properly. Applying positive control spots for data correction away from the spot needed to calibrated not only adds unnecessary operations but also requires an extra area for spots, causing lowering of the array density.

An alternative method has been reported that a nucleic acid probe and a labeled nucleic acid, both of which are labeled with fluorescent dyes having fluorescent resonance energy transfer, are reacted to form a hybrid on the same spot, and the fluorescent intensity value of the reaction system, in which the target nucleic acid and the probe nucleic acid are hybridized, is calibrated with the fluorescent intensity value of the un-hybridized probe nucleic acid, the fluorescent intensity value of un-hybridized target nucleic acid and the fluorescent intensity value of the target nucleic acid by fluorescent resonance energy transfer (Patent reference 1). However, since this method requires fluorescent intensity measurements before and after the hybrid formation, there is a problem of the probe detachment after the hybrid formation during the washing operation and the like. There is also a problem that the combination of labels is limited to the one having fluorescent resonance energy transfer and further that it appears to be difficult to carry out the hybrid formation to completion in a microarray using protein, because the phenomenon of fluorescent resonance energy transfer occurs in the distance of 1-9 bases and it is difficult to label protein at a specific site.

### SUMMARY OF THE INVENTION

An object of the present invention is to provide an analytical method and an analytical instrument with high capability of quantitative determination and reproducibility. By using these, the analytical method of the present invention can be applied for the detection of fine difference in expression level, the measurement of protein concentration and the like.

The present invention provides an analytical method for biological samples, which makes accurate measurements of the quantity and the like of the target biological sample possible, comprising the steps of: fixing a target biological sample and a fixing agent which fixes the target biological sample onto the same spot; labeling the target biological sample and the fixing agent, or the fixing agent with a plurality of labels in which no resonance energy transfer occurs, and the wavelength of light signals thereof do not overlap; and measuring the intensity of light after the formation of the bound material of the target biological sample. The present invention also provides an analytical instrument, which is suitable for carrying out this analytical method, and a microarray.

The present invention corrects the signal from the label for analysis by receiving the signals from a plurality of spots of materials for correction fixed onto a microarray or the like and extracting the characteristics of the signals. It is also possible to calibrate the light intensity of the label for analysis by carrying out spotting (fixation of biological samples to a microarray) on a different day and educing the characteristic amount of the label for correction.

According to the present invention an analytical method and an analytical instrument with good capability of quantitative determination and reproducibility are provided which may be utilized for detecting fine differences in expression level and for measuring protein concentration and the like.

### BRIEF DESCRIPTION OF THE DRAWINGS

FIG. 1 is a schematic view of sandwich immunoassay;

FIG. 2 is a flow chart for analysis in the Embodiment 1; and

FIG. 3 is a schematic diagram of the analytical instrument of biological sample of the present invention.

### PREFERRED EMBODIMENTS OF THE INVENTION

In the present invention, fluorescent, enzyme and RI (radio isotope) labels may be utilized, and in the following description fluorescent labels are mainly used.

The present invention provides an analytical method and analytical instrument for biological samples, wherein two or more labels, in which no resonance energy transfer occurs and the maximum wavelength of light signals do not overlap, are present at the same position in a microarray, and the fluorescent intensity of the one specific label is used to calibrate the signal intensity of the other labels. Also provided are a microarray and an immunoassay suitable for the method and the instrument described above.

According to an embodiment of the present invention, an analytical method for biological samples are provided, wherein two or more of different labels, in which no mutual resonance energy transfer occurs, are fixed in the same area of an immunoassay in which the target biological sample is fixed onto a support using a fixing agent, and the signal intensity of a specific label is used to normalize the signal intensity of other labels. The normalizing method preferably uses statistical values such as median or mean.

According to the embodiment, of the present invention, an analytical method for biological samples are provided, wherein two or more different fluorescent labels, in which no mutual fluorescent resonance energy transfer occurs, are fixed in the same area of an immunoassay in which the target biological sample is fixed onto a support using a fixing agent, and the signal intensity of a specific fluorescent label is used to normalize the signal intensity of other fluorescent labels. There are a plurality of fluorescent labels, and the first fluorescent material is for correction and the second fluorescent material is for analytical use.

The immunoassay described above is any of:

(1) an immunoassay comprising a target biological sample fixed onto a support using a fixing agent, the first fluorescent material bound to the fixing agent described above and the second fluorescent material bound to the biological sample described above; (2) an immunoassay comprising a target biological sample fixed onto a support using a first fixing agent, a first fluorescent material bound to the first fixing agent described above, a second fixing agent bound to the biological sample described above, and a second fluorescent material bound to the second fixing agent described above, and (3) an immunoassay comprising a target biological sample fixed onto a support using the first fixing agent, the first fluorescent material bound to the first fixing agent described above, the second fixing agent bound to the biological sample described above, the third fixing agent bound to the second fixing agent described above and the second fluorescent material bound to the third fixing agent described above. It is preferable that the difference in the fluorescent wavelength among a plurality of fluorescent materials is 30 nm or greater. Further the support for fixing preferably includes slide glass, a silicon substrate, a microtiter well, fine particles of silica, fine particles of metal, a gel, membrane or PDMS. Still further, the fixing agent by which the fluorescent label is fixed to the substrate may be a nucleic acid, a PNA, an antibody, an aptamer, an antigen, a protein, a low molecular weight substance or a substitute thereof. It is preferable that the base material, on which the immunoassay described above is fixed, is a microchip.

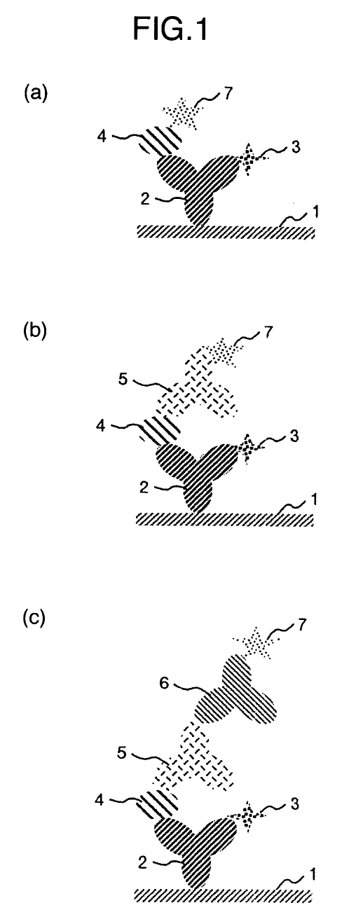

In the present invention, following 3 typical immunoassays are used.
(1) An immunoassay for analyzing biological samples comprising a first binder 2, such as an antibody and the like, bound to a microarray base material 1, a biological sample 4 bound to the first binder described above, a label for correction 3, for example a fluorescent label, bound to the first binder described above and a label for quantitative determination 7, for example a fluorescent label, bound to the biological sample described above, wherein no mutual resonance energy transfer occurs between the label for correction and the label for quantitative determination described above (FIG. 1A).
(2) An immunoassay for analyzing biological samples comprising a first binder 2 bound to a microarray base material 1, a biological sample 3 bound to the first binder described above, a label for correction 3 bound to the first binder described above, a second binder 5 bound to the biological sample described above and a label for quantitative determination 7 bound to the second binder described above, wherein no mutual resonance energy transfer occurs between the label for correction and the label for quantitative determination described above (FIG. 1B).
(3) An immunoassay for analyzing biological samples comprising a first binder 2 bound to a microarray base material 1, a biological sample 4 bound to the first binder described above, a label for correction 3 bound to the first binder described above, a second binder 5 bound to the biological sample described above and a third binder 6 bound to the second binder described above and a label for quantitative determination 7 bound to the third binder described above (FIG. 1C).

The immunoassay used in the present invention is used most reasonably as a microarray described below. It is also preferable to make a microchip by adding channels and flow cells as necessary.

A microarray having an immunoassay selected from the group consisting of: (1) an immunoassay comprising a target biological sample fixed onto a support through a fixing agent, a first label bound to the fixing agent described above and a second labels bound to the biological sample described above; (2) an immunoassay comprising a target biological sample fixed onto a support through a first fixing agent, a first label bound to the first fixing agent described above, a second fixing agent bound to the biological sample described above and a second label bound to the second fixing agent described above; and (3) an immunoassay comprising a target biological sample fixed onto a support through a first fixing agent, a first label bound to the first fixing agent described above, a second fixing agent bound to the biological sample described above, a third fixing agent bound to the second fixing agent described above and a second label bound to the third fixing agent described above, wherein no mutual resonance energy transfer occurs between the first label and the second label described above.

Further, the present invention provides an instrument for analyzing a biological sample comprising: a device which irradiates with electromagnetic waves which excite each of a plurality of labels of the microarray; a detector which detects light signals emitted by the irradiation; and a computer which normalizes and corrects the light signal of the second label described above by the signal of the first label described above. When RI labels are used, the device which irradiates with electromagnetic waves is not required.

The labels may be used in the present invention include materials shown in Table 1. Two or more materials, in which the difference of the wavelength is 30 nm or greater, are selected from the materials in the Table and are used as a fluorescent label for analysis or as a label for calibrating the fluorescent intensity of the fluorescent label for analysis described above.

TABLE 1

| | Excitation | Fluorescence |
|---|---|---|
| Fluorescent label | wave length | wave length |
| ALEXA 350 | 346 | 442 |
| PACIFIC BLUE | 416 | 451 |
| MARINA BLUE | 362 | 459 |
| ACRIDINE | 362 | 462 |
| EDANS | 336 | 468 |
| COUMARIN | 432 | 472 |
| BODIPY 493/503 | 493 | 503 |
| CY2 | 489 | 506 |
| BODIPY FL-X | 504 | 510 |
| DANSYL | 335 | 518 |
| ALEXA 488 | 495 | 519 |
| FAM | 495 | 520 |
| OREGON GREEN | 500 | 520 |
| RHODAMINE GREEN-X | 503 | 528 |
| NBD-X | 466 | 535 |
| TET | 521 | 536 |
| ALEXA 430 | 434 | 541 |
| BODIPY R6G-X | 529 | 547 |
| JOE | 520 | 548 |
| ALEXA 532 | 532 | 554 |
| VIC | 538 | 554 |
| HEX | 535 | 556 |
| CAL ORANGE | 524 | 557 |
| ALEXA 555 | 555 | 565 |
| BODIPY 564/570 | 563 | 569 |
| BODIPY TMR-X | 544 | 570 |
| QUASAR 570 | 550 | 570 |
| ALEXA 546 | 556 | 573 |
| TAMRA | 555 | 576 |
| PHODAMINE RED-X | 560 | 580 |
| BODIPY 581/591 | 581 | 591 |
| CY3.5 | 581 | 596 |
| ROX | 575 | 602 |
| ALEXA 568 | 578 | 603 |
| CAL RED | 583 | 603 |
| BODIPY TR-X | 588 | 616 |
| ALEXA 594 | 590 | 617 |
| BODIPY 630/650-X | 625 | 640 |
| PULSAR 650 | 460 | 650 |
| BODIPY 630/665-X | 646 | 660 |
| ALEXA 647 | 650 | 665 |
| QUASAR 670 | 649 | 670 |

### EXAMPLES

The present invention will be explained in detail with following examples.

### Example 1

A microarray measurement by the first embodiment of the present invention will be explained based on the antigen-antibody reaction of interleukin 8. FIG. 1C is a schematic view of sandwich immunoassay used in the present example. Reference numeral 1 denotes a support on which protein and the like are fixed, and the slide glass for protein microarray (Proteochip TR) used as the support 1 may be purchased from Hitachi High-Technologies Co.

In addition to slide glass, silicon substrate, microtiter well, fine particles of silica, fine particles of gold, a gel, a membrane, PDMS and the like may be used as the support 1.

Further, functional groups such as amino group, aldehyde group, epoxy group and the like may be introduced to the surface of the support 1 using various chemicals. Still further, functional groups may be fixed onto gel, polymer, membrane and the like. In the case of the particle- or powder-form support, the support is added to the solution containing biological sample and reacted to fix the biological sample on the support.

Devices for microarray production, which is called arrayers or spotters, are commercially. available. These devices prepare an array on a substrate such as slide glass and the like by disposing many spots in a matrix form with a needle on which the solution is attached or by micro-spraying with an ink-jet nozzle which sucks in the solution. And then an array is produced by drying the solution. Usually the gap between the spots is 100-1,000 μm and the distance between the neighboring spots is 100-1,000 μm. Therefore, one microarray using slide glass can accommodate from several 1000s to several 10,000s spots.

For evaluating the antigen-antibody reaction of interleukin 8 (sandwich immunoassay), following reagents were used: anti-interleukin 8 monoclonal antibody (Fitzgerald Co.) as a primary antibody 2 fixed onto the support 1; Recombinant Human interleukin 8 (Serotec Co.) as an antigen 4; anti-interleukin 8 polyclonal antibodies (Fitzgerald Co.) as a secondary antibody 5; Cy5 Goat Anti-Rabbit IgG (H+L) Conjugate (Zymed Laboratories Co.) as a tertiary antibody 6. The label for quantitative determination 7 (Cy5) is the tertiary antibody 6 labeled with fluorescent label. The primary antibody 2 may be substituted with nucleic acid, PNA, antibody, aptamer, antigen, protein, low molecular weight substance or their substitutes.

The fluorescent label for data correction 3 (Cy3) is the primary antibody 2 labeled with fluorescent label using FluoroLink-Ab Cy3 Labeling Kit (manufactured by Amersham Pharmacia Biotech). Fluorescent labeling is completed in about 2 hours using this labeling kit. The excitation wavelength and fluorescent wavelength of Cy3 are 552 nm and 565 nm, respectively. The excitation wavelength and fluorescent wavelength of Cy5 are 650 nm and 667 nm, respectively. It is preferable that there is no fluorescence resonance energy transfer between the fluorescent labels 3 and 7, and that the difference of the maximum wavelength is 30 nm or more. In addition to fluorescent label, the fluorescent labels 3 and 7 may be labeled with enzyme or RI (radioisotope).

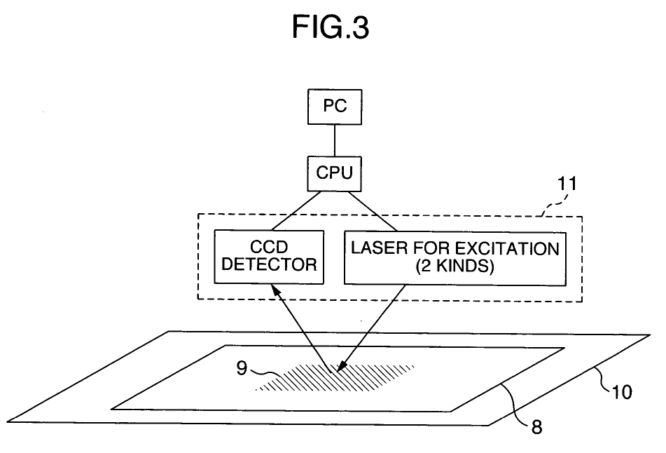

The primary antibody 2 was prepared by mixing Cy3 labeled anti-interleukin 8 monoclonal antibody and non-labeled anti-interleukin 8 monoclonal antibody at the ratio of 1:100 and diluting with PBS (phosphate buffered saline, pH 7.4) containing 30% glycerol to a final concentration of 100 μg/ml, and spotted at 1.5 μl per spot. The mixing ratio of Cy3 labeled anti-interleukin 8 monoclonal antibody and non-labeled anti-interleukin 8 monoclonal antibody is adjusted so that the signal intensity is within the dynamic range of a fluorescent scanner 11 of FIG. 3.

In the present embodiment the appropriate mixing ratio was in the range of 1:10,000-1:1. Further, it is not necessarily required to use Cy3 labeled anti-interleukin 8 monoclonal antibody, but a mere fluorescent material such as fluorescently labeled protein A may be spotted. This is because the function of the fluorescent label for data correction 3 is only to calibrate the fluorescent intensity of the fluorescent label for quantitative determination 7. Thus detection of the fluorescent labels 3 and 7 may be carried out in different conditions.

The spotted chips were incubated overnight in a 37° C. incubator to fix and then blocked for 1 hour with gentle stirring in PBS (pH 7.4) containing 3% BSA (bovine serum albumin) to prevent non-specific absorption in the following steps.

And then chips were washed in washing solution (a) [PBS (pH 7.8) containing 0.5% Tween 20 (surface active agent, Sigma Co.)] for 5 minutes and the washing is repeated 3 times. Further, the antigen was diluted with dilution solution (b) [PBS (pH 7.4) containing 1% BSA and 30% glycerol] to an appropriate concentration and spotted at 1.5 μl per each spot. The spotted chips were incubated in a 37° C. incubator for 1 hour.

After that, chips were washed with the washing solution (a) for 5 minutes and the washing was repeated 3 times. Further, the secondary antibody was diluted with the dilution solution (b) to 1:100 and spotted at 1.5 μl per each spot. After incubating spotted chips in the 37° C. incubator for 30 minutes, the chips were washed with the washing solution (a) for 5 minutes and the washing is repeated 3 times. Further, the tertiary antibody was diluted with the dilution solution (b) to 1:200 and spotted at 1.5 μl per each spot. The spotted slide were incubated in the 37° C. incubator for 30 minutes and then the chips were washed 3 times for 5 minutes per washing with the washing solution (a). Finally chips were dried under a stream of nitrogen.

In measuring the fluorescent intensity of labels 3 and 7 spotted on a slide glass substrate, both Cy3 and Cy5 were detected using a fluorescent scanner with 20 μm resolution (ScanArray Express, Packard BioScience Co.) under a condition of laser intensity 80%, and photomultiplier intensity 80%. However, since the fluorescent intensity Cy3 of 3 is used only for calibrating the fluorescent intensity of Cy5 of 7, the detection conditions, such as the laser power and photomultiplier intensity, for Cy3 and Cy5 may be different, unlike in the method for expression analyses which compares the fluorescence in the same condition.

In the detection of 3 and 7, Cy3 was excited at 543 nm and measured light at 565 nm, and Cy5 is excited at 633 nm and measured light at 667 nm. Each signal intensity and the background are converted to numerical values using internal algorithm of QuantArray (Packard BioScience Co.), which is the attached software for the fluorescent scanner. In the present embodiment, median values are used as signal intensity but other statistical values such as mean and the like may be used. After converting to numerical values, the efficiency of the microarray of the present invention is evaluated by comparing CV value (standard deviation/mean×100) of the signal intensity value of Cy5 without correction and that with correction with the signal intensity of Cy3.

Table 2 shows means, standard deviations and CV values of 4 spots when the concentration of the antigen 4 is changed and at each concentration 4 spots are measured. (1) shows fluorescent intensity data of the label for quantitative determination 7 (conventional method), (2) shows fluorescent intensity data of the fluorescent label for data correction 3, (3) shows (1) data corrected by (2) data. In (1), CV values of Cy5 data is 10% or above at the antigen concentration of 0-0.1 ng/ml, but by applying the correction using the Cy3 data of (2) of the present invention, all of the CV values of Cy5 data after correction shown in (3) are 5% or less. These suggest that the signal intensities in lane 4 in Table 2 (1), (2) and (3) are calibrated appropriately.

[Table 2]

TABLE 2

| Antigen | | | | | | | |
|---|---|---|---|---|---|---|---|
| concentration | Lane | | | | | Standard | |
| (ng/ml) | 1 | 2 | 3 | 4 | Mean | deviation | CV value |

[(1) Cy5 data]

| | | | | | | | |
|---|---|---|---|---|---|---|---|
| 3 | 845 | 760 | 855 | 909 | 842 | 62 | 7.3 |
| 1 | 817 | 795 | 823 | 860 | 824 | 27 | 3.3 |
| 0.1 | 775 | 777 | 828 | 611 | 748 | 94 | 12.6 |
| 0.01 | 857 | 830 | 808 | 520 | 754 | 157 | 20.8 |
| 0 | 851 | 826 | 830 | 435 | 736 | 201 | 27.3 |

[(2) Cy3 data]

| | | | | | | | |
|---|---|---|---|---|---|---|---|
| 3 | 37890 | 36399 | 39803 | 43998 | 39523 | 3293 | 8.3 |
| 1 | 36181 | 36628 | 38551 | 44483 | 38961 | 3822 | 9.8 |
| 0.1 | 36855 | 35777 | 37881 | 28291 | 34701 | 4359 | 12.6 |
| 0.01 | 40178 | 39270 | 39019 | 22710 | 35294 | 8404 | 23.8 |
| 0 | 39102 | 40352 | 41573 | 20271 | 35325 | 10086 | 28.6 |

[(3) Cy5 data after correction]

| | | | | | | | |
|---|---|---|---|---|---|---|---|
| 3 | 819 | 766 | 789 | 758 | 783 | 27 | 3.5 |
| 1 | 829 | 797 | 784 | 764 | 794 | 27 | 3.4 |
| 0.1 | 772 | 797 | 802 | 793 | 791 | 13 | 1.7 |
| 0.01 | 783 | 776 | 760 | 841 | 790 | 35 | 4.5 |
| 0 | 799 | 751 | 733 | 788 | 768 | 31 | 4.0 |

The reason for the improvement of the CV values is thought to be because the influence such as warping of the slide glass, unevenness of linker coating, detachment of probe during washing of the slide glass and the like may have been corrected by the correction and the cause may be determined by observing the interference pattern of the slide glass and the like. The normalization of Cy5 data by Cy3 data is carried out by the following formula.
Intensity of the Cy5 signal (each spot)=Intensity of the Cy5 signal before correction (each spot)×[Mean of intensity of the Cy3 signal (20 spots)/Intensity of the Cy3 signal (each spot)]

Here, if the condition may be different, for example the degree of quenching of the fluorescent label at different antigen concentration is different and the like, the correction may be carried out by calculating the mean value at each antigen concentration.

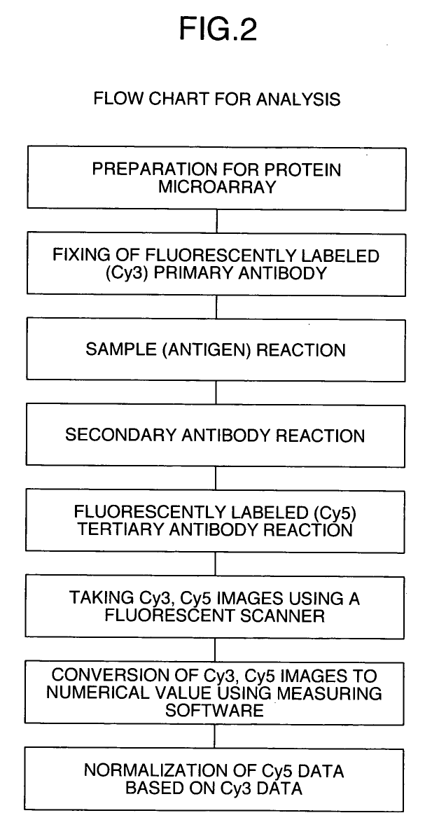

According to the embodiment of the present invention, the microarray chips with a labeled primary antibody may be supplied, and the contract analyses using these chips may be carried out. FIG. 2 shows a flow chart for the analysis in Embodiment 1, and FIG. 3 is a schematic diagram of the analytical instrument for biological sample according to the present invention. In FIG. 3, slide glass 8 is mounted on a slide glass stage 10, and the microarray spots 9, on which the immunoassay is fixed, are formed on the surface of the slide glass 8. A plurality of labels are fixed onto the immunoassay, and by irradiating laser light with suitable excitation wavelength for each label, the fluorescent signal from the label is detected by a CCD detector. The labels are selected so that the difference in fluorescent wavelength is 30 nm or greater. The signal from the one label is used for calibrating the signal from the other label (for quantitative determination or detection).

1. A method for analyzing a biological sample comprising the steps of: fixing two or more different labels, among which no mutual resonance energy transfer occurs, to the same area of immunoassay where a target biological sample is fixed to a support using a fixing agent; generating light signals by exciting the labels; and normalizing, using the signal intensity of one specific label, the signal intensity of the other labels.

2. The method for analyzing a biological sample according to claim 1, wherein the label is a fluorescent label, enzyme label or RI label.

3. The method for analyzing a biological sample according to claim 1, comprising using an immunoassay, wherein a target biological sample and a label for correction are fixed to a fixing agent, and a label for analysis are fixed onto the same spot using one or more fixing agents.

4. The method for analyzing a biological sample according to claim 1, wherein a first label is for correction and a second label is for analysis.

5. The method for analyzing a biological sample according to claim 1, wherein the immunoassay is any of: (1) an immunoassay comprising a target biological sample fixed onto a support through a fixing agent, a first label bound to the fixing agent and a second label bound to the biological sample; (2) an immunoassay comprising a target biological sample fixed onto a support through a first fixing agent, a first label bound to the first fixing agent, a second fixing agent bound to the biological sample and a second label bound to the second fixing agent; and (3) an immunoassay comprising a target biological sample fixed onto a support through a first fixing agent, a first label bound to the first fixing agent, a second fixing agent bound to the biological sample, a third fixing agent bound to the second fixing agent and a second label bound to the third fixing agent.

6. The method for analyzing a biological sample according to claim 1, wherein the difference in the fluorescent wavelength among a plurality of fluorescent labels is 30 nm or greater.

7. The method for analyzing a biological sample according to claim 1, wherein the support for fixing includes slide glass, a silicon substrate, a microtiter well, fine particles of silica, fine particles of gold, a gel, a membrane or PDMS (polydimethylsiloxane).

8. The method for analyzing a biological sample according to claim 1, wherein the fixing agent for fixing a label onto a support is a nucleic acid, a PNA, an antibody, an aptamer (a nucleic acid with binding activity that is a DNA or RNA molecule which binds to a specific target molecule), an antigen, a protein, a low molecular weight substance or a substitute thereof.

9. The method for analyzing a biological sample according to claim 1, wherein statistical values such as median values or mean values are used for normalization.

10. The method for analyzing a biological sample according to claim 1, wherein a base material onto which the immunoassay is fixed is a microchip.

11. An immunoassay for analyzing a biological sample comprising a first binder bound to a microarray, a biological sample bound to the first binder, a label for correction bound to the first binder, and a label for quantitative determination bound to the biological sample, wherein the label for correction and the label for quantitative determination do not cause any mutual resonance energy transfer.

12. An immunoassay for analyzing a biological sample comprising a first binder bound to a microarray, a biological sample bound for the first binder, a label for correction bound to the first binder, a second binder bound to the biological sample, and a label for quantitative determination bound to the second binder, wherein the label for correction and the label for quantitative determination are substances in which no mutual resonance energy transfer occurs.

13. An immunoassay for analyzing a biological sample comprising a first binder bound to a microarray, a biological sample bound for the first binder, a label for correction bound to the first binder, a second binder bound to the biological sample, a third binder bound to the second binder and a label for quantitative determination bound to the third binder, wherein the label for correction and the label for quantitative determination are substances in which no mutual resonance energy transfer occurs.

14. A microarray having on a base material an immunoassay, which is any of: (1) an immunoassay comprising a target biological sample fixed onto a support through a fixing agent, a first label bound to the fixing agent and a second label bound to the biological sample; (2) an immunoassay comprising a target biological sample fixed onto a support through a first fixing agent, a first label bound to the first fixing agent, a second fixing agent bound to the biological sample and a second label bound to the second fixing agent; and (3) an immunoassay comprising a target biological sample fixed onto a support through a first fixing agent, a first label bound to the first fixing agent, a second fixing agent bound to the biological sample, a third fixing agent bound to the second fixing agent and a second label bound to the third fixing agent, wherein the first label and the second label do not cause any mutual resonance energy transfer.

15. An instrument for analyzing a biological sample comprising a microarray according to claim 14, a device which irradiates with electromagnetic waves that excite each of a plurality of labels of the microarray, a detector which detects light signals emitted by the irradiation and a computer which normalizes and corrects the light signal of the second label by the signal of the first label.
